(19) United States
(12) Patent Application Publication
(10) Pub. No.: US 2010/0292575 A1
(43) Pub. Date: Nov. 18, 2010

(54) DEVICE AND METHOD FOR IDENTIFYING TAMPERING OF AN ULTRASOUND PROBE

(75) Inventor: Gabriel Keith Sharp, Charlestown, IN (US)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY, Schenectady, NY (US)

(21) Appl. No.: 12/466,446

(22) Filed: May 15, 2009

(51) Int. Cl. A61B 8/14

(52) U.S. Cl. 600/459

(57) ABSTRACT

An ultrasound probe includes a housing having an interior chamber formed therein. The ultrasound probe also includes an electronics assembly provided in the housing, the electronics assembly converting acoustic energy to electrical signals, and a tamper indicating substance applied to the ultrasound probe, the tamper indicating substance adapted to provide a visual indication when the ultrasound probe has been tampered with. A system including the ultrasound probe and a method of fabricating the ultrasound probe are also provided.

### BACKGROUND OF THE INVENTION

This invention relates generally to ultrasound systems, and more particularly, to a device and method for determining whether an ultrasound probe has been subjected to an unauthorized repair, or abuse with the intent to claim warranty entitlement, or mishandling.

Ultrasound systems typically include ultrasound scanning devices, such as, ultrasound probes having different control components and transducers that allow for performing various different ultrasound scans (e.g., different imaging of a volume or body). These ultrasound probes may include control components within different portions of the probe, including, for example, the probe handle and the probe connection member for connecting to an ultrasound system. These control components within the probe allow for controlling operation of the probe by an ultrasound system, for example, to operate in different modes, such as, amplitude mode (A-mode), brightness mode (B-1 mode), power Doppler mode, color imaging mode, among others.

Ultrasound probes are manufactured by a company referred to herein as the original equipment manufacture (OEM). To manufacture the ultrasound probe, and to receive regulatory approval to supply the probe to an end user, the OEM is required to comply with the regulatory guidelines set forth in ISO 10993 for the evaluation and testing of medical devices. ISO 10993 generally provides that the OEM can not install any parts or material into an ultrasound probe, used in clinical applications, that may have toxicity or other side effects, such as latex, for example. After the OEM receives regulatory approval from the FDA, the ultrasound probe is supplied to the end user.

In some cases, the regulatory compliance liability continues to reside with the OEM even after the ultrasound probe is supplied to the end user. That is, the OEM may be responsible to ensure that the ultrasound probe complies with ISO 10993 after being supplied to the end user. As such, the OEM may be responsible for ensuring that the ultrasound probe complies with ISO 10993 throughout the operational life of the ultrasound probe.

During normal use, the ultrasound probe may be damaged and thus require repair or replacement of various ultrasound probe parts. In some cases, the end user returns the ultrasound probe to the OEM for repair. The OEM then utilizes repair components and materials to ensure that the ultrasound probe complies with ISO 10993. However, in other cases, the end user may send the ultrasound probe to an unauthorized repair facility. In this case, the unauthorized repair facility may use repair parts that do not comply with ISO 10993. Moreover, unauthorized repair facilities may perform minor repairs and then present the repaired ultrasound probe as factory new, i.e. repaired to the same specifications as required by the OEM, or new, i.e. manufactured by the OEM and never placed in operation. In either case, the unauthorized repair facility may not have performed biocompatibility testing on materials or parts being used to repair the ultrasound probe. Biocompatibility testing may include, for example, testing the ultrasound probe to ensure that the replacement parts and/or materials have the same acoustic properties as the original parts and/or materials and are approved under ISO 10993. For example, the speed of sound through the non-OEM repaired ultrasound probe may not produce the same image quality or results as an OEM repaired ultrasound probe. The non-OEM repaired ultrasound probe may result in a reduction in patient safety or a product quality issue for the OEM.

It is difficult for the OEM to determine whether an ultrasound probe has been repaired by an authorized repair facility or the OEM. Moreover, it is difficult for the OEM to determine whether an unauthorized repair facility has previously performed repairs on the ultrasound probe. Thus, current ultrasound probe designs may not adequate to enable the OEM to determine whether a previously repaired ultrasound probe meets the requirements of ISO 10993 or if the probe is entitled to warranty service under the terms and conditions stated by the OEM.

### BRIEF DESCRIPTION OF THE INVENTION

In one embodiment an ultrasound probe is provided. The ultrasound probe includes a housing having an interior chamber formed therein. The ultrasound probe also includes an electronics assembly provided in the housing, the electronics assembly converting acoustic energy to electrical signals, and a tamper indicating substance applied to the ultrasound probe, the tamper indicating substance adapted to provide a visual indication when the ultrasound probe has been tampered with, intentionally abused or mishandled.

In another embodiment, an ultrasound imaging system is provided. The ultrasound imaging system includes a beamformer and an ultrasound probe. The ultrasound probe includes a housing having an interior chamber formed therein. The ultrasound probe also includes an electronics assembly provided in the housing, the electronics assembly converting acoustic energy to electrical signals, and a tamper indicating substance applied to the ultrasound probe, the tamper indicating substance adapted to provide a visual indication when the ultrasound probe has been tampered, intentionally abused or mishandled.

In yet another embodiment, a method for fabricating an ultrasound probe is provided. The method includes applying a tamper indicating substance to the ultrasound probe, the tamper indicating substance adapted to provide a visual indication when the ultrasound probe has been tampered with, intentionally abused or mishandled.

### BRIEF DESCRIPTION OF THE DRAWINGS

FIG. 1 is a block diagram of an ultrasound system in accordance with an exemplary embodiment of the present invention.

FIG. 2 is a block diagram of an ultrasound system in accordance with another exemplary embodiment of the present invention.

FIG. 3 is a perspective view of an image of an object acquired by the systems of FIGS. 1 and 2 in accordance with an exemplary embodiment of the present invention.

FIG. 4 is a side elevation view of an exemplary ultrasound probe constructed in accordance with an embodiment of the invention.

FIG. 5 is an exploded view of the ultrasound probe shown in FIG. 4 in accordance with an embodiment of the invention.

### DETAILED DESCRIPTION OF THE INVENTION

Exemplary embodiments of ultrasound probes providing for the detection of tampering, modification, adulteration, and/or ultrasound probe abuse and mishandling are described in detail below. In particular, a detailed description of exemplary ultrasound systems is first provided followed by a detailed description of various embodiments of ultrasound probes.

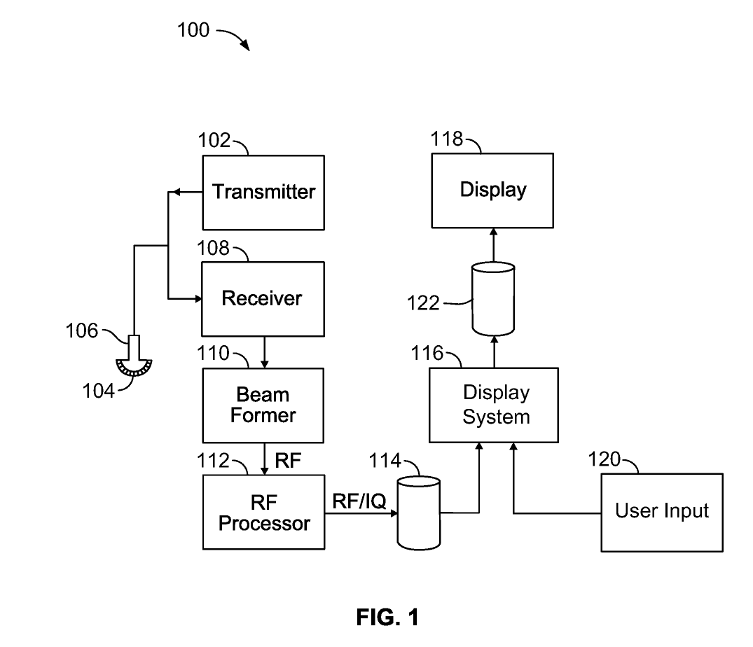

FIG. 1 illustrates a block diagram of an exemplary embodiment of an ultrasound system 100 that may be used, for example, to acquire and process ultrasonic images. The ultrasound system 100 includes a transmitter 102 that drives an array of elements 104 (e.g., piezoelectric crystals) within or formed as part of a transducer 106 to emit pulsed ultrasonic signals into a body or volume. A variety of geometries may be used and one or more transducers 106 may be provided as part of a probe (not shown). The pulsed ultrasonic signals are back-scattered from density interfaces and/or structures, for example, in a body, like blood cells or muscular tissue, to produce echoes that return to the elements 104. The echoes are received by a receiver 108 and provided to a beamformer 110. The beamformer 110 performs beamforming on the received echoes and outputs an RF signal. The RF signal is then processed by an RF processor 112. The RF processor 112 may include a complex demodulator (not shown) that demodulates the RF signal to form IQ data pairs representative of the echo signals. The RF or IQ signal data then may be routed directly to an RF/IQ buffer 114 for storage (e.g., temporary storage).

The ultrasound system 100 also includes a signal processor 116 to process the acquired ultrasound information (i.e., RF signal data or IQ data pairs) and generate frames of ultrasound information for display on a display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 114 during a scanning session and processed in less than real-time in a live or off-line operation.

The ultrasound system 100 may continuously acquire ultrasound information at a frame rate that exceeds fifty frames per second, which is the approximate perception rate of the human eye. The acquired ultrasound information is displayed on the display system 118 at a slower frame-rate. An image buffer 122 may be included for storing processed frames of acquired ultrasound information that are not scheduled to be displayed immediately. In an exemplary embodiment, the image buffer 122 is of sufficient capacity to store at least several seconds of frames of ultrasound information. The frames of ultrasound information may be stored in a manner to facilitate retrieval thereof according to their order or time of acquisition. The image buffer 122 may comprise any known data storage medium.

A user input device 120 may be used to control operation of the ultrasound system 100. The user input device 120 may be any suitable device and/or user interface for receiving user inputs to control, for example, the type of scan or type of transducer to be used in a scan.

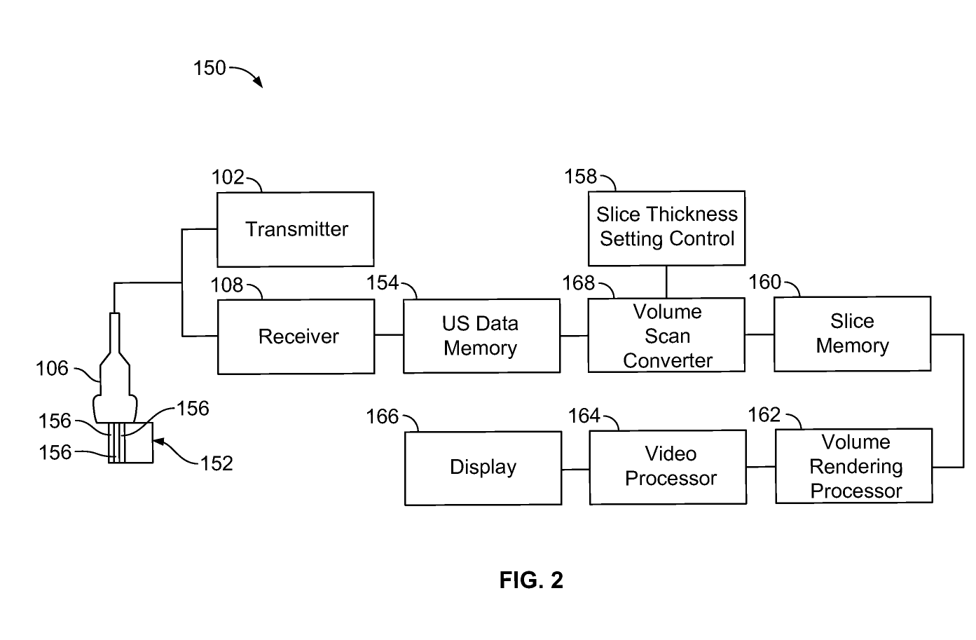

FIG. 2 illustrates a block diagram of another exemplary embodiment of an ultrasound system 150 that may be used, for example, to acquire and process ultrasonic images. The ultrasound system 150 includes the transducer 106 in communication with the transmitter 102 and receiver 108. The transducer 106 transmits ultrasonic pulses and receives echoes from structures inside a scanned ultrasound volume 152. A memory 154 stores ultrasound data from the receiver 108 derived from the scanned ultrasound volume 152. The scanned ultrasound volume 152 may be obtained by various techniques, including, for example, 3D scanning, real-time 3D imaging, volume scanning, scanning with transducers having positioning sensors, freehand scanning using a Voxel correlation technique, 2D scanning or scanning with a matrix of array transducers, among others.

The transducer 106 is moved, such as along a linear or arcuate path, while scanning a region of interest (ROI). At each linear or arcuate position, the transducer 106 obtains a plurality of scan planes 156. The scan planes 156 are collected for a thickness, such as from a group or set of adjacent scan planes 156. The scan planes 156 are stored in the memory 154, and then provided to a volume scan converter 168. In some exemplary embodiments, the transducer 106 may obtain lines instead of the scan planes 156, with the memory 154 storing lines obtained by the transducer 106 rather than the scan planes 156. The volume scan converter 168 receives a slice thickness setting from a slice thickness setting control 158, which identifies the thickness of a slice to be created from the scan planes 156. The volume scan converter 168 creates a data slice from multiple adjacent scan planes 156. The number of adjacent scan planes 156 that are obtained to form each data slice is dependent upon the thickness selected by the slice thickness setting control 158. The data slice is stored in a slice memory 160 and accessed by a volume rendering processor 162. The volume rendering processor 162 performs volume rendering upon the data slice. The output of the volume rendering processor 162 is provided to a video processor 164 that processes the volume rendered data slice for display on a display 166.

It should be noted that the position of each echo signal sample (Voxel) is defined in terms of geometrical accuracy (i.e., the distance from one Voxel to the next) and one or more ultrasonic responses (and derived values from the ultrasonic response). Suitable ultrasonic responses include gray scale values, color flow values, and angio or power Doppler information.

It should be noted that the ultrasound systems 100 and 150 may include additional or different components. For example, the ultrasound system 150 may include a user interface or user input 120 (shown in FIG. 1) to control the operation of the ultrasound system 150, including, to control the input of patient data, scan parameters, a change of scan mode, and the like.

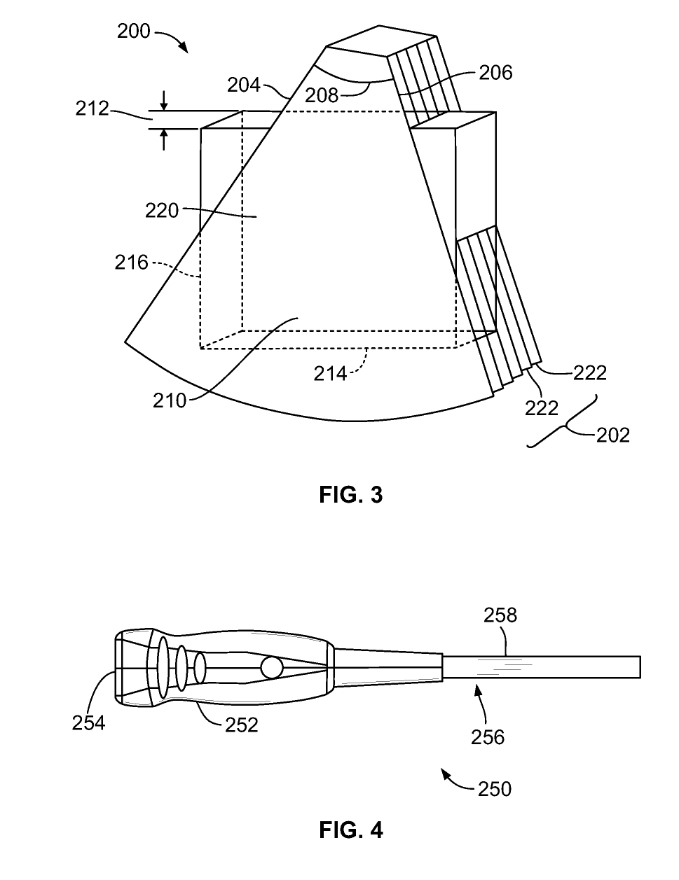

FIG. 3 illustrates an exemplary image of an object 200 that may be acquired by the ultrasound systems 100 and 150. The object 200 includes a volume 202 defined by a plurality of sector shaped cross-sections with radial borders 204 and 206 diverging from one another at an angle 208. The transducer 106 (shown in FIGS. 1 and 2) electronically focuses and directs ultrasound firings longitudinally to scan along adjacent scan lines in each scan plane 156 (shown in FIG. 2) and electronically or mechanically focuses and directs ultrasound firings laterally to scan adjacent scan planes 156. The scan planes 156 obtained by the transducer 106, and as illustrated in FIG. 1, are stored in the memory 154 and are scan converted from spherical to Cartesian coordinates by the volume scan converter 168. A volume comprising multiple scan planes 156 is output from the volume scan converter 168 and stored in the slice memory 160 as a rendering region 210. The rendering region 210 in the slice memory 160 is formed from multiple adjacent scan planes 156.

The rendering region 210 may be defined in size by an operator using a user interface or input to have a slice thickness 212, width 214 and height 216. The volume scan converter 168 (shown in FIG. 2) may be controlled by the slice thickness setting control 158 (shown in FIG. 2) to adjust the thickness parameter of the slice to form a rendering region 210 of the desired thickness. The rendering region 210 defines the portion of the scanned ultrasound volume 152 that is volume rendered. The volume rendering processor 162 accesses the slice memory 160 and renders along the slice thickness 212 of the rendering region 210.

Referring now to FIGS. 1 and 2, during operation, a slice having a pre-defined, substantially constant thickness (also referred to as the rendering region 210) is determined by the slice thickness setting control 158 and is processed in the volume scan converter 168. The echo data representing the rendering region 210 (shown in FIG. 3) may be stored in the slice memory 160. Predefined thicknesses between about 2 mm and about 20 mm are typical, however, thicknesses less than about 2 mm or greater than about 20 mm may also be suitable depending on the application and the size of the area to be scanned. The slice thickness setting control 158 may include a control member, such as a rotatable knob with discrete or continuous thickness settings.

The volume rendering processor 162 projects the rendering region 210 onto an image portion 220 of an image plane(s) 222 (shown in FIG. 3). Following processing in the volume rendering processor 162, pixel data in the image portion 220 may be processed by the video processor 164 and then displayed on the display 166. The rendering region 210 may be located at any position and oriented at any direction within the volume 202. In some situations, depending on the size of the region being scanned, it may be advantageous for the rendering region 210 to be only a small portion of the volume 202.

FIG. 4 is a perspective view of an exemplary ultrasound probe 250 constructed in accordance with an exemplary embodiment of the invention. The ultrasound probe 250 generally includes a housing 252 having a scan portion 254 and a connection portion 256. The housing 252 generally includes therein control components and operating components for performing ultrasound scans. For example, and in general, the housing 252 may include therein a transducer array (not shown) having a plurality of elements, such as, for example, piezoelectric elements (not shown) and control components, for example, electrical components mounted to a printed circuit board (not shown). The scan portion 254 is used to scan, for example, a patient, by emitting therefrom ultrasonic waves and receiving echoes as is known. The connection portion 256 includes a system cable 258 for connection to, for example, an ultrasound system scanning controller via a connection (not shown) as is known.

It should be noted that the ultrasound probe 250 may include additional component parts, for example, a control knob (not shown) that is rotatable between an engaged and a disengaged position to control operation of the ultrasound probe 250. In the exemplary embodiment, the ultrasound probe may be utilized with imaging system 100, imaging system 150, or any other ultrasound imaging system.

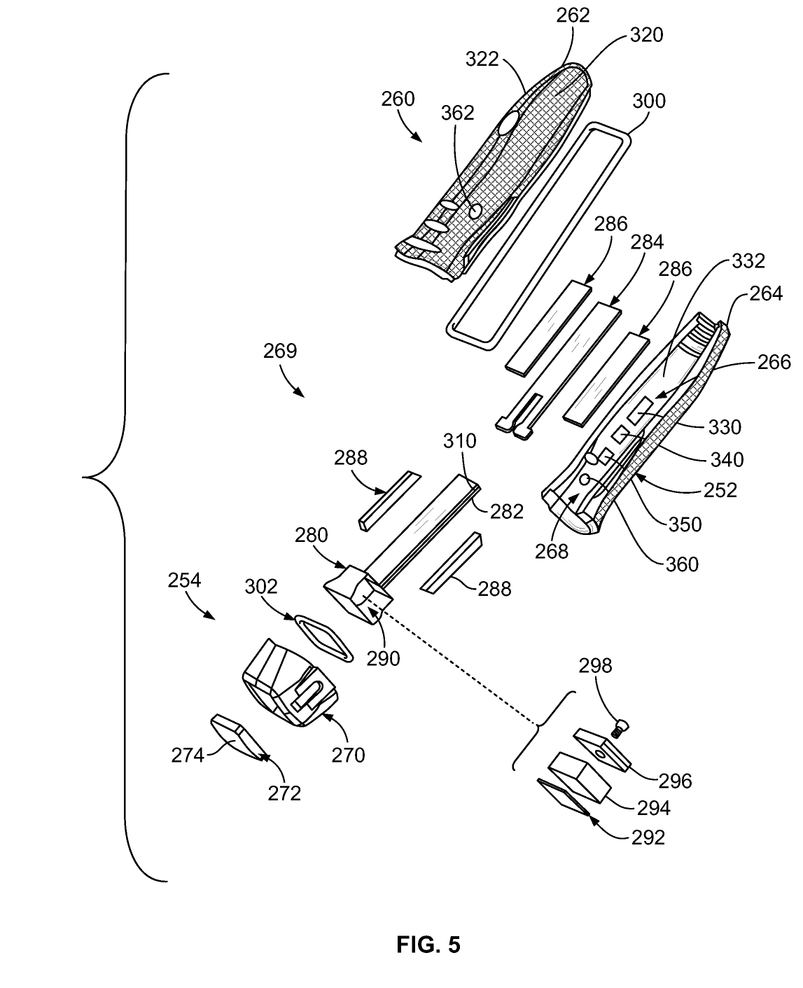

FIG. 5 is an exploded view of the ultrasound probe 250 shown in FIG. 4. As discussed above, the ultrasound probe 250 includes the housing 252, which in this embodiment is formed in as a multi-piece apparatus and generally includes a handle portion 260 including a first handle portion 262 and a second handle portion 264. During assembly, the first and second handle portions 262 and 264 are coupled together to form the handle portion 260. In the exemplary embodiment, the first and second handle portions 262 and 264 are secured together using screws, adhesive, and/or other compounds and form an interior chamber 266 having an open front end 268. The interior chamber 266 may be configured to receive therein, for example, an electronics subassembly 269. The probe 250 at the scan portion 254 generally includes a nosepiece 270 (having a recess on a back side thereof) and a lens assembly 272. In one exemplary embodiment, the lens assembly 272 includes a lens 274 formed of silicon.

The ultrasound probe electronics assembly 269 includes at least a connection member 280, which in one embodiment is a flexible printed circuit board. The connection member 280 may be formed of multiple layers, and include a portion 282 for receiving therebetween a plate 284. The connection member 280 may be connected to the plate 284, for example, using pressure sensitive adhesive tape 286. Connectors 288 also may be provided as part of the connection member 280 for interfacing and connection therewith. The connection member 280 also may form an opening 290 for receiving therein a ceramic composite 292, a backing strip 294 and a block 296, together forming a transducer assembly as is known. A screw 298 or other securing member also may be provided for connecting or securing the various components together.

As discussed above, it is difficult for the OEM to determine whether an ultrasound probe has been tampered with. Tampering may include, for example, having the ultrasound probe repaired by a repair facility or other personnel that have not been authorized by the OEM. Tampering may also include modifying the ultrasound probe and/or installing unauthorized components in the ultrasound probe, e.g. the probe is adulterated. Tampering may also include cleaning and/or disinfecting the ultrasound probe with unauthorized solutions or caustic cleaners, and/or intentionally mishandling the ultrasound probe by dropping or striking the ultrasound probe, for example. Tampering may also include subjecting the ultrasound probe to extreme temperatures. Each of the above conditions may require the ultrasound probe to be repaired. Described herein are methods and apparatuses to enable the OEM or OEM authorized repair personnel to determine whether the ultrasound probe has been tampered with.

In the exemplary embodiment, the ultrasound probe 50 includes at least one of several features that are adapted to provide a visual indication to the OEM, or an OEM authorized repair facility, that the ultrasound probe has been tampered with. Unauthorized, as used herein, describes either a repair facility or other personnel that are not the OEM or authorized by the OEM to repair ultrasound probes in accordance with the OEM guidelines and/or approved ISO guidelines.

In one embodiment, the ultrasound probe 250 includes a tamper indicating substance that is applied to, or affixed to, at least a portion of the ultrasound probe 250 that provides a visual indication that the ultrasound probe 250 has been tampered with. In one exemplary embodiment, the tamper indicating substance is a fluorescing dye that fluoresces when exposed to ultraviolet (UV) light. Moreover, when the dye is not exposed to UV light, the dye has a clear or opaque appearance. In the exemplary embodiment, the tamper indicating substance, e.g. the fluorescing dye, fluoresces a predetermined color when exposed to UV light. For example, as discussed above, it is difficult for the OEM to determine when an authorized repair or tampering has occurred with the ultrasound probe. In the exemplary embodiment, the fluorescing dye provides a visual indication of potential tampering. In one embodiment, the color of the fluorescing dye is determined by the OEM. One example of a fluorescing dye is 109-MSK-UR manufactured by the DYMAX Company. Therefore, the OEM can easily determine whether the fluorescing dye has been partially removed or altered by an unauthorized repair facility thus providing a visual indication to the OEM that an unauthorized repair has been performed on the ultrasound probe 250.

As discussed above, tampering may include, for example, having the ultrasound probe repaired by a repair facility or other personnel that have not been authorized by the OEM. To perform this repair, the unauthorized repair facility typically disassembles the ultrasound probe 250. To determine when the ultrasound probe 250 has been at least partially disassembled, in one embodiment, the tamper indicating substance includes at least one seal that includes the fluorescing dye. For example, referring again to FIG. 5, in the one embodiment, the ultrasound probe 250 includes a seal 300.

The seal 300 is disposed between the first and second handle portions 262 and 264. In the one embodiment, the seal 300 is a compound that includes an adhesive and the fluorescing dye. The adhesive is adapted to provide a seal at the seam line formed between the first and second handle portions 262 and 264. The fluorescing dye is adapted to provide a visual indication when the seal 300 has been broken or altered. For example, when the seal 300 is installed by the OEM, the seal 300 will fluoresce the predetermined color when exposed to UV light. However, if the seal 300 is broken or replaced, the replacement seal will not fluoresce the predetermined color. The seal 300 enables the OEM to easily identify that the seal 300 has been broken or replaced during an unauthorized repair procedure. As shown in FIG. 5, the ultrasound probe 250 may also includes a seal 302. The seal 302 is substantially the same as the seal 300 and performs the same functions.

It should be realized that although only two seals are illustrated in FIG. 5, ultrasound probe 250 may include multiple seals. More specifically, the ultrasound probe 250 may include multiple bond lines, e.g. seams or points where two parts are coupled together. A seal having an adhesive material and the fluorescing dye may be used at each seam or bond line to fabricate the probe 250.

Other evidence may be utilized by the OEM when the ultrasound probe 250 has been at least partially disassembled, or the seals have been broken. In one embodiment, the tamper indicating substance includes a moisture indicating substance that is applied to at least a portion of the interior surface of the ultrasound probe 250. More specifically, when an ultrasound probe has been improperly repaired, the OEM may identify this repair based on moisture present within the interior of the ultrasound probe 250. Referring again to FIG. 5, in the one embodiment, the ultrasound probe 250 includes a moisture indicating label 330 that is affixed to an interior surface 332 of the ultrasound probe 250. The moisture indicating label 330 includes the tamper indicating substance.

In this embodiment, the tamper indicating substance is a substance that is adapted to change from a first color to a different second color when the moisture within the ultrasound probe 250 exceeds a predetermined threshold. For example, during normal use, when little or no moisture is present within the ultrasound probe, the moisture indicating label 330 is a first color such as white, for example. However, if the moisture exceeds the predetermined threshold, the moisture indicating label 330 appears as a different second color, such as red, for example. The moisture indicating label 330 enables the OEM to easily identify if at least one seal on the ultrasound probe 50 has been breached for unauthorized repair based on the color of the moisture indicating label 330. Thus the moisture indicating label 330 provides a visual indication of an unauthorized repair procedure.

In another embodiment, the tamper indicating substance is embodied as a chemical indicating substance that is applied to at least a portion of the interior surface of the ultrasound probe 250. More specifically, when an ultrasound probe is cleaned using an unauthorized cleaning solution, the OEM may identify this solution based on a color of the chemical indicating substance. Referring again to FIG. 5, in the one embodiment, the ultrasound probe 250 includes a moisture chemical indicating label 340 that is affixed to either the interior surface 332 of the ultrasound probe 250 or the exterior surface 322 of the ultrasound probe 250. The chemical indicating label 340 includes the tamper indicating substance. In this embodiment, the tamper indicating substance is a substance that is adapted to change from a first color to a different second color when a predetermined chemical contacts the chemical indicating label. For example, during approved use, when the ultrasound probe 250 is cleaned using an authorized cleaning solution, the chemical indicating label 340 is a first color such as white, for example. However, if the ultrasound probe 250 is cleaned using an unauthorized cleaning solution, that breaches the seals, the chemical indicating label 340 appears as a different second color, such as red, for example.

The chemical indicating label 340 enables the OEM to easily identify when at least one seal on the ultrasound probe 50 has been breached using an unauthorized cleaning solution based on the color of the chemical indicating label 340. Thus the chemical indicating label 340 provides a visual indication of an unauthorized repair procedure. In one embodiment, the chemical indicating label 340 may include a plurality of chemical indicating substances each adapted to turn from a first color to a different second color based on the type of chemical that label 340 is exposed to. The chemical indicating label provides the OEM with an indication of the particular type of chemical that the ultrasound probe 250 was subjected to.

As discussed above, tampering may include, for example, modifying the ultrasound probe and/or installing unauthorized components in the ultrasound probe, e.g. the probe is adulterated. More specifically, unauthorized repair facilities may break the housing seals, as discussed above, and then replace authorized parts with unauthorized parts. To determine when the ultrasound probe 250 has been adulterated, in one embodiment, the tamper indicating substance includes a conformal coating that is applied to at least a portion of the probe 250. For example, referring again to FIG. 5, in one embodiment, the ultrasound probe 250 includes a conformal coating 310 that is applied to a surface of the electronics assembly 269. Conformal coating, as used herein, is a coating that adheres to and follows the contours of the object to which the coating is applied. For example, circuit boards typically include a substrate and numerous electronic devices coupled to and extending outwardly from the substrate, thus creating a non-uniform surface. During fabrication, the conformal coating 310 is applied to a surface, such as the surface of electronics assembly 269, and follows the contours of both the substrate and the electronic devices coupled to the substrate.

For example, to repair an electronic circuit, such as electronics assembly 269, the unauthorized repair facility may remove the entire circuit board and or at least some electronic devices installed on the circuit board. To determine when the ultrasound probe 250 adulterated, the tamper indicating substance, e.g. the fluorescing dye is applied as the conformal coating 310.

In one embodiment, the conformal coating 310 may be applied to any structure or device of ultrasound probe 250. In use, the conformal coating 310 is applied by the OEM and fluoresces the predetermined color when exposed to UV light. However, if the electronics assembly 269 or any electronic device installed on the electronics assembly 269 is replaced, the replacement devices will not fluoresce the predetermined color. Therefore, the OEM can easily identify that at least a portion of the conformal coating 310 is missing from certain devices thus providing a visual indication of an unauthorized repair procedure.

It should be realized that although only one conformal coating 310 is illustrated in FIG. 5, that other portions of the probe 250 may include a conformal coating. More specifically, a conformal coating may be applied to any portion of the probe 250 which the OEM desires to determine prior tampering. For example, a conformal coating may be applied to connectors such as connection member 280 and/or connector 288.

As discussed above, tampering may also include, for example, cleaning the ultrasound probe 250 using an unauthorized cleaning solution or solvent. More specifically, personnel may utilize a caustic substance to sterilize or disinfect the ultrasound probe 250. The caustic substance may cause damage to the seals as discussed above.

To determine when the ultrasound probe 250 has been subjected to an unauthorized cleaning solution, in one embodiment, the tamper indicating substance includes a conformal coating that is applied to at least a portion of the exterior surface of the ultrasound probe 250. For example, referring again to FIG. 5, in one embodiment, the ultrasound probe 250 includes a conformal coating 320 that is applied to an exterior surface 322 of the ultrasound probe housing 252. More specifically, the tamper indicating substance is the conformal coating 320 that includes the fluorescing dye. As discussed above, the conformal coating 320 adheres to and follows the contours of the object to which the coating is applied. In this example, the conformal coating 330 adheres to and follows the contours of the exterior surface 322 of the ultrasound probe 250.

In use, the conformal coating 320 is applied by the OEM and fluoresces the predetermined color when exposed to UV light. However, if the ultrasound probe 250 is subjected to an unauthorized cleaning solution, the conformal coating 320 is removed and the exposed portions of the ultrasound probe 250 will not fluoresce the predetermined color. The conformal coating 320 enables the OEM to easily identify that certain areas of the conformal coating 320 are missing thus providing a visual indication of an unauthorized use of a cleaning solution.

In another embodiment, the conformal coating 320 is not utilized, rather the tamper indicating substance that includes the fluorescing dye is formed as part of the ultrasound housing 252 itself. More specifically, the fluorescing dye may be included as part of the compound used to form the housing 252. In one embodiment, the tamper indicating material is integral to the housing 252.

To determine when the ultrasound probe 250 has been subjected to an unauthorized cleaning solution, in one embodiment, when the ultrasound probe 250 is subjected to an unauthorized cleaning solution, the integral dye is warn away or removed and the exposed portions of the ultrasound probe 250 will not fluoresce the predetermined color. The conformal coating 320 enables the OEM to easily identify that the either portions of the housing 252 have been replaced by unauthorized portions or that certain areas of the ultrasound probe 250 have been cleaned using an unauthorized cleaning solution thus providing a visual indication of an unauthorized use of a cleaning solution.

It should be realized that although only one conformal coating 310 is illustrated in FIG. 5, that other portions of the probe 250 may include a conformal coating. More specifically, a conformal coating may be applied to any portion of the probe 250 which the OEM desires to determine prior tampering. For example, a conformal coating may be applied to connectors such as connection member 280 and/or connector 288.

In another embodiment, to determine when the ultrasound probe 250 has been subjected to extreme temperatures, the tamper indicating substance is embodied as a temperature indicating label 350 that is affixed to the interior surface 332 of the ultrasound probe 250. More specifically, the temperature indicating label 350 includes the tamper indicating substance. In this embodiment, the tamper indicating substance is a substance that is adapted to change from a first color to a different second color when an internal temperature of the ultrasound probe 250 exceeds a predetermined threshold. For example, during normal use, the ultrasound probe 250 operates a temperature that is less than the predetermined threshold and the temperature indicating label 350 is a first color such as white, for example. However, if an internal temperature of the ultrasound probe 250 exceeds the predetermined threshold, the temperature indicating label 350 appears as a different second color, such as red, for example. The temperature indicating label 350 enables the OEM to easily identify when the ultrasound probe 250 has been subjected to inappropriate temperatures based on the color of the temperature indicating label 350.

In another embodiment, to determine when the ultrasound probe 250 has been subjected to a shock event that may effect the operation of the ultrasound probe 250, the ultrasound probe 250 further includes at least one shock sensor 360. In one embodiment, the shock sensor 360 is installed in the interior chamber 266 and emits a signal when a shock event exceeds a predetermined threshold. The shock sensor 360 may be embodied as a passive shock sensor. The shock sensor 360 may emit a visual indication or an electric signal that may be obtained by the OEM by disassembling the ultrasound probe 250. For example, the passive shock sensor 360 may emit a light signal or other indication that is only observable during disassembly. Optionally, the shock sensor 360 may be embodied as an active shock sensor that emits a signal that is observable without disassembling the ultrasound probe 250. For example, the ultrasound probe 250 may include a visual indication or LED 362 that is coupled to the shock sensor 360. In use, when the ultrasound probe 250 is subjected to a shock event, the shock sensor 360 transmits a signal to the LED 362 thus illuminating the LED. The shock sensor 360 provides a visible indication to the OEM that a shock event has occurred. Moreover, the shock sensor may also provide, via the LED 362, a visual indication to an operator that a shock event has occurred. In either case, the shock sensor provides a visual indication that potential damage has occurred to the ultrasound probe 250.

Described herein is a system and method for detection of tampering, modification, adulteration, and product abuse and mishandling, either incidental or intentional of an ultrasound probe. Each component of the system and method provides visual and indisputable evidence to customers, field service personnel, and make centers of tampering.

Thus, various embodiments of the present invention provide an ultrasound probe having a tamper indicating substance applied thereto to provide a visual indication when the ultrasound probe has been tampered with. The ultrasound probe may include a biocompatible fluorescing UV Coating and bond line adhesive. This coating is applied to probe seamlines, membranes, gasket areas and the plastic shell of the ultrasound probes. During use, the UV coating fluoresces red due when exposed to any UV light source. The coating is also be clear and does not change the chemical or biocompatibility structure of any existing resins or epoxies utilized in manufacturing the ultrasound probe.

The ultrasound probe may also include micro self-contained shock sensors that are placed within the probe body to detect drops or impacts of 10G or greater. The shock sensors may be used as a verification to initial observed physical damage or as a validation measure for suspected physical damage. In one embodiment, the shock sensors are tubular micro shock sensors, self-contained, and indicate excessive shock by turning the entire tube red, and have a response time that is less than 50 ms.

The ultrasound probe may also include moisture, chemical, and/or temperature detecting labels. The pre-printed detection labels easily identify fluid ingress issues and temperature issues and determine warranty entitlement or a compromise to IPX7 rating. In one embodiment, the detecting labels change in color from white to red when exposed to high levels of moisture or temperature.

As a result, the ultrasound probe described herein provide product authentication. That is the ultrasound probe described herein includes features that enable the OEM to uniquely and indisputably identify a genuine and unadulterated ultrasound probe. Therefore, the ultrasound probe described herein reduces warranty entitlement and fraudulent warranty claims, identify 3rd Party probe repair adulteration and unauthorized modifications, identify customer mishandling of the ultrasound probes, provide visual evidence based evaluation of probe failure modes, and also provide a decentralization of warranty entitlement decisions. It should be noted that the various embodiments of probes described herein are not limited to a particular application, but may be used in different applications as desired or needed, for example, in medical imaging, non-destructive testing and/or sonar evaluation.

While the invention has been described in terms of very-specific embodiments, those skilled in the art will recognize that the invention can be practiced with modification within the spirit and scope of the claims.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

What is claimed is:

1. An ultrasound probe, comprising:
an ultrasound probe housing having an interior chamber formed therein;
an electronics assembly provided in said housing, said electronics assembly converting acoustic energy to electrical signals; and
a tamper indicating substance applied to the ultrasound probe, said tamper indicating substance adapted to provide a visual indication when the ultrasound probe has been tampered with.

2. An ultrasound probe in accordance with claim 1, wherein said housing comprises:
a first portion;
a second portion coupled to said first portion; and
a seal disposed between said first and second portions, said seal comprising said tamper indicating substance.

3. An ultrasound probe in accordance with claim 1, wherein said tamper indicating substance comprises a fluorescing material adapted to fluoresce when exposed to ultraviolet light.

4. An ultrasound probe in accordance with claim 1, wherein said tamper indicating substance comprises a fluorescing dye adapted to fluoresce a predetermined color when exposed to ultraviolet light.

5. An ultrasound probe in accordance with claim 1, wherein said tamper indicating substance comprises a fluorescing dye adapted to fluoresce a predetermined color when exposed to ultraviolet light, the predetermined color indicating a manufacturer of the ultrasound probe.

6. An ultrasound probe in accordance with claim 1, wherein said tamper indicating substance comprises a sealing compound and a fluorescing dye mixed with said sealing compound, said sealing compound adapted to seal said housing portions, said fluorescing dye adapted to fluoresce a predetermined color when exposed to ultraviolet light.

7. An ultrasound probe in accordance with claim 1, wherein said tamper indicating substance comprises a conformal ultraviolet coating applied to an exterior surface of said housing and adapted to fluoresce when exposed to ultraviolet light.

8. An ultrasound probe in accordance with claim 1, wherein said tamper indicating substance comprises a conformal ultraviolet coating applied to an exterior surface of said electronics assembly and adapted to fluoresce when exposed to ultraviolet light.

9. An ultrasound probe in accordance with claim 1, wherein said tamper indicating substance comprises a moisture indicating substance that is disposed on a moisture detecting label that is affixed to said ultrasound probe.

10. An ultrasound probe in accordance with claim 1, wherein said tamper indicating substance comprises a chemical indicating substance that is disposed on a chemical detecting label that is affixed to the ultrasound probe.

11. An ultrasound probe in accordance with claim 1, further comprising at least one shock sensor disposed in said interior chamber, said shock sensor adapted to determine when the ultrasound probe is subjected to a shock event that is exceeds a predetermined threshold.

12. An ultrasound system comprising:
a beamformer; and
an ultrasound probe coupled to said beamformer, said ultrasound probe comprising
a housing having an interior chamber formed therein;
an electronics assembly provided in said housing, said electronics assembly converting acoustic energy to electrical signals; and
a tamper indicating substance applied to said ultrasound probe, said tamper indicating substance adapted to provide a visual indication when said ultrasound probe has been tampered with.

13. An ultrasound system in accordance with claim 12, wherein said ultrasound probe comprises a seal comprising said tamper indicating substance.

14. An ultrasound system in accordance with claim 12, wherein said tamper indicating substance comprises a fluorescing dye adapted to fluoresce a predetermined color when exposed to ultraviolet light.

15. An ultrasound system in accordance with claim 12, wherein said tamper indicating substance comprises a conformal ultraviolet coating applied to at least one of an exterior surface of said housing and an exterior surface of said electronics assembly and adapted to fluoresce when exposed to ultraviolet light.

16. An ultrasound system in accordance with claim 12, wherein said tamper indicating substance comprises at least one of a moisture indicating substance, temperature indicating substance and a chemical indicating substance that is disposed on a label that is affixed to an interior portion of said ultrasound probe.

17. An ultrasound system in accordance with claim 12, wherein said ultrasound probe further comprises at least one shock sensor disposed in said interior chamber, said shock sensor adapted to determine when said ultrasound probe is subjected to a shock event that is exceeds a predetermined threshold.

18. A method of fabricating an ultrasound probe, said method comprising applying a tamper indicating substance to said ultrasound probe, said tamper indicating substance adapted to provide a visual indication when said ultrasound probe has been tampered with.

19. A method in accordance with claim 18, further comprising applying a tamper indicating substance that includes a fluorescing dye adapted to fluoresce a predetermined color when exposed to ultraviolet light to the ultrasound probe.

20. A method in accordance with claim 18, further comprising applying at least one of conformal coating, a moisture indicating substance, a temperature indicating substance, and a chemical indicating substance to an interior portion of the ultrasound probe.

21. A method in accordance with claim 18, further comprising coupling at least one shock sensor in the interior chamber of the ultrasound probe, the shock sensor adapted to determine when the ultrasound probe is subjected to a shock event that is exceeds a predetermined threshold.
